(19) United States
(12) Patent Application Publication
(10) Pub. No.: US 2011/0293479 A1
(43) Pub. Date: Dec. 1, 2011

(54) CARTRIDGE FOR MEASURING BIOLOGICAL SAMPLE COMPONENT AND APPARATUS FOR MEASURING BIOLOGICAL SAMPLE COMPONENT

(75) Inventors: Jong Cheol HONG, Daejeon (KR); Chul Huh, Daejeon (KR); Bong Kyu Kim, Daejeon (KR); Chil Seong Ah, Daejeon (KR); Yo Han Choi, Daejeon (KR); Gun Yong Sung, Daejeon (KR)

(73) Assignee: Electronics and Telecommunications Research Institute, Daejeon (KR)

(21) Appl. No.: 12/950,964

(22) Filed: Nov. 19, 2010

(30) Foreign Application Priority Data
May 25, 2010 (KR) 10-2010-0048812

(51) Int. Cl. G01N 21/00; G01N 33/48

(52) U.S. Cl. 422/82.05; 422/68.1

(57) ABSTRACT

A cartridge for measuring a biological sample component and an apparatus for measuring a biological sample component are provided. The cartridge for measuring a biological sample component includes: an upper cartridge having a first chamber storing a biological sample, a first channel connected to the first chamber and delivering air pressure to the first chamber, and a third channel transferring the biological sample; and a lower cartridge having a third chamber storing a first reagent, a fifth chamber storing a second reagent, a fifth channel connecting the third and fifth chambers and transferring the second reagent to the third chamber, an eleventh channel delivering the biological sample delivered through the third channel to the third chamber, and a thirteenth channel delivering air pressure to the fifth chamber, wherein when the upper and lower cartridges are bounded by an external force, the third channel and the eleventh channel are connected.

### CROSS-REFERENCE TO RELATED APPLICATIONS

This application claims the priority of Korean Patent Application No. 10-2010-0048812 filed on May 25, 2010, in the Korean Intellectual Property Office, the disclosure of which is incorporated herein by reference.

### BACKGROUND OF THE INVENTION

1. Field of the Invention

The present invention relates to a measurement of a biological sample component and, more particularly, to a cartridge for measuring a biological sample component and an apparatus for measuring a biological sample component.

2. Description of the Related Art

The generally used disposable medical diagnosis tools transfer and diagnose a biological sample mainly by using capillary action (or capillarity). However, the medical diagnosis tools using capillary action largely use a diagnostic reagent in a dried state, rather than in a liquid state, and because they are available merely to manipulate a fluid in a simple form, the medical diagnosis tools are largely employed for qualitative checking.

For example, the measurement of the amount of microalbumin in urine is essential for monitoring a chronic renal disease (or kidney ailments), but, at the same time, the amount of creatinine must also be measured to correct the measurement results, which thus requires a complicated manipulation of fluid, and because the diagnostic reagent used for the measurement is in a liquid state, a diagnosis tool using capillary action cannot be used. Thus, in order to measure the amount of microalbumin and creatinine in urine, a diagnosis tool capable of actively manipulating a fluid and transferring and mixing a desired reagent and a sample is required.

### SUMMARY OF THE INVENTION

An aspect of the present invention provides a cartridge for measuring a biological sample component by easily transferring and mixing a diagnostic reagent.

Another aspect of the present invention provides an apparatus for measuring a biological sample component by using the cartridge for measuring a biological sample component.

According to an aspect of the present invention, there is provided a cartridge for measuring a biological sample component, including: an upper cartridge having a first chamber storing a biological sample, a first channel connected to the first chamber and delivering air pressure to the first chamber, and a third channel transferring the biological sample; and a lower cartridge having a third chamber storing a first reagent, a fifth chamber storing a second reagent, a fifth channel connecting the third and fifth chambers and transferring the second reagent to the third chamber, an eleventh channel delivering the biological sample delivered through the third channel to the third chamber, and a thirteenth channel delivering air pressure to the fifth chamber, wherein when the upper and lower cartridges are bound by an external force, the third channel and the eleventh channel are connected.

The lower cartridge may further include a ninth channel connected to the first channel when the lower cartridge is bound to the upper cartridge, in order to provide air pressure provided from the exterior to the first channel.

First and third pump holes may be prepared in one side of the ninth channel and in one side of the thirteenth channel and be connected to an external pump, respectively, in order to be provided with air pressure therethrough, respectively.

The lower cartridge may further include a first air exhaust hole connected to the third chamber to exhaust air present in the third chamber and a seventh channel connecting the first air exhaust hole and the third chamber.

The upper cartridge may further include a second chamber storing the biological sample, a second channel connected to the second chamber to deliver air pressure to the second chamber, and a fourth channel transferring the biological sample, and the lower cartridge may further include a fourth chamber storing a third reagent, a sixth chamber storing a fourth reagent, a sixth channel connecting the fourth and sixth chambers and transferring the fourth reagent to the fourth chamber, a twelfth channel delivering the biological sample delivered through the fourth channel to the fourth chamber, and a fourteenth channel delivering air pressure to the sixth chamber.

The lower cartridge may further include a tenth channel connected to the second channel when the lower cartridge is bound to the upper cartridge to provide air pressure provided from the exterior to the second channel.

Second and fourth pump holes may be prepared in one side of the tenth channel and in one side of the fourteenth channel and be connected to an external pump, respectively, in order to be provided with air pressure, respectively.

The lower cartridge may further include a second air exhaust hole connected to the fourth chamber and exhausting air present in the fourth chamber, and a sixth channel connecting the second air exhaust hole and the fourth chamber.

According to another aspect of the present invention, there is provided an apparatus for measuring a biological sample component using a cartridge storing a biological sample and at least one reagent and a plurality of channels transferring the biological sample and the at least one reagent and delivering air pressure, including: a first pump providing a first air pressure for transferring a biological sample stored in a first chamber of the cartridge to a third chamber of the cartridge, to a channel connected to the first chamber; a third pump providing a second air pressure for transferring a second reagent stored in a fifth chamber of the cartridge to a third chamber of the cartridge, to a channel connected to the fifth chamber; a first light emitting unit emitting light to a mixture stored in the third chamber; a first light receiving unit detecting the amount of light which has transmitted through the mixture stored in the third chamber and providing the detected amount of light; and a controller controlling the driving of the first and third pumps to allow the biological sample and the at least one reagent to be mixed in the third chamber, and acquiring the density of a first component of the biological sample.

The controller may control the driving of the third pump to transfer the second reagent stored in the fifth chamber to the third chamber to mix the second reagent and the first reagent stored in the third chamber, and then control the driving of the first light emitting unit to emit light to the third chamber and set a first amount of transmitted light provided from the first light receiving unit as a first reference value for measuring the density of the first component of the biological sample.

After setting the first reference value, the controller may control the driving of the first pump to transfer the biological sample stored in the first chamber to the third chamber to mix the biological sample and the mixture which has been obtained by mixing the first and second reagents, and then control the driving of the first light emitting unit to emit light to the third chamber, acquire a second amount of transmitted light provided from the first light emitting unit, and measure the density of the first component by comparing the acquired second amount of transmitted light and the first reference value.

The biological sample may be a urine sample, the first reagent may be picric acid, the second reagent may be an aqueous sodium hydroxide solution, and the first component may be creatinine.

The apparatus may further include: a second pump providing a third air pressure, which is for transferring the biological sample stored in the second chamber of the cartridge to the fourth chamber of the cartridge, to a channel connected to the second chamber; a fourth pump providing a fourth air pressure, which is for transferring the third reagent stored in the sixth chamber to the fourth chamber of the cartridge, to a channel connected to the sixth chamber; a second light emitting unit emitting light to a mixture stored in the fourth chamber; and a second light receiving unit detecting the amount of light which has transmitted through the mixture stored in the fourth chamber and providing the detected amount of light, wherein the controller controls the driving of the second and fourth pumps to allow the biological sample and the at least one reagent to be mixed in the fourth chamber, and acquires the density of a second component of the biological sample.

The controller may control the driving of the second pump to transfer the biological sample stored in the second chamber to the fourth chamber to mix the biological sample and the third reagent stored in the fourth chamber, and then control the driving of the second light emitting unit to emit light to the fourth chamber and set a third amount of transmitted light provided from the second light receiving unit as a second reference value for measuring the density of the second component of the biological sample.

After setting the second reference value, the controller may control the driving of the fourth pump to transfer the fourth reagent stored in the sixth chamber to the fourth chamber to mix the fourth reagent and the mixture which has been obtained by mixing the biological sample and the third reagent, and then control the driving of the second light emitting unit to emit light to the fourth chamber, acquire a fourth amount of transmitted light provided from the second light emitting unit, and measure the density of the second component by comparing the acquired fourth amount of transmitted light and the second reference value.

The biological sample may be a urine sample, the third reagent may be an aqueous polyvinylpyrolidone (PVP) solution, the fourth reagent may be an antibody directed against microalbumin, and the second component may be microalbumin.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a view illustrating the structure of a cartridge for measuring a biological sample component according to an exemplary embodiment of the present invention;

FIG. 2 is a view illustrating the structure of a cartridge for measuring a biological sample component according to another exemplary embodiment of the present invention;

FIG. 3 is a schematic block diagram of the apparatus for measuring a biological sample component according to an exemplary embodiment of the present invention;

FIG. 4 is a flow chart illustrating the process of a method for measuring a biological sample component according to an exemplary embodiment of the present invention; and

FIG. 5 is a flow chart illustrating the process of a method for measuring a biological sample component according to another exemplary embodiment of the present invention.

### DETAILED DESCRIPTION OF THE PREFERRED EMBODIMENT

The present invention may be modified variably and may have various embodiments, particular examples of which will be illustrated in drawings and described in detail.

However, it should be understood that the following exemplifying description of the invention is not intended to restrict the invention to specific forms of the present invention but rather the present invention is meant to cover all modifications, similarities and alternatives which are included in the spirit and scope of the present invention.

While terms such as “first” and “second,” etc., may be used to describe various components, such components must not be understood as being limited to the above terms. The above terms are used only to distinguish one component from another. For example, a first component may be referred to as a second component without departing from the scope of rights of the present invention, and likewise a second component may be referred to as a first component. The term “and/or” encompasses both combinations of the plurality of related items disclosed and any item from among the plurality of related items disclosed.

When a component is mentioned as being “connected” to or “accessing” another component, this may mean that it is directly connected to or accessing the other component, but it is to be understood that another component may exist therebetween. On the other hand, when a component is mentioned as being “directly connected” to or “directly accessing” another component, it is to be understood that there are no other components in-between.

The terms used in the present application are merely used to describe particular embodiments, and are not intended to limit the present invention. An expression used in the singular encompasses the expression of the plural, unless it has a clearly different meaning in the context. In the present application, it is to be understood that terms such as “including” or “having,” etc., are intended to indicate the existence of the features, numbers, operations, actions, components, parts, or combinations thereof disclosed in the specification, and are not intended to preclude the possibility that one or more other features, numbers, operations, actions, components, parts, or combinations thereof may exist or may be added.

Unless otherwise defined, all terms used herein, including technical or scientific terms, have the same meanings as those generally understood by those with ordinary knowledge in the field of art to which the present invention belongs. Such terms as those defined in a generally used dictionary are to be interpreted as having meanings equal to the contextual meanings in the relevant field of art, and are not to be interpreted as having ideal or excessively formal meanings unless clearly defined as having such in the present application.

Embodiments of the present invention will be described below in detail with reference to the accompanying drawings, where those components that are the same or are in correspondence are rendered using the same reference number, regardless of the figure number, and redundant explanations are omitted.

Hereinafter, in an exemplary embodiment of the present invention, a cartridge for measuring a biological sample component and an apparatus for measuring a biological sample component are used to measure the density of microalbumin and creatinine components in urine. However, the cartridge for measuring a biological sample component and the apparatus for measuring a biological sample component are not limited to the measurement of the density of microalbumin and creatinine but may be used to measure the density of various components included in a biological sample.

The density of the microalbumin component in urine is measured by using a reaction of an antibody directed against polyvinylpyrolidone nano-particles and microalbumin. Namely, the presence of microalbumin in a urine sample causes the PVP nano-particles and the antibody directed against microalbumin to react to microalbumin, increasing absorbance in a particular wavelength.

In measuring the density of microalbumin in urine, first, the PVP nano-particles and a urine sample are mixed by using the foregoing characteristics, absorbance is measured and set as a reference value, and an antibody is added to the mixed sample, and a change in the absorbance is then measured, to thus measure the density of the microalbumin component.

In addition, the density of creatinine in urine is measured by using a reaction utilizing picric acid. Namely, the picric acid has a yellowish dark red color in reaction to creatinine in an alkaline solution, so the density of creatinine is measured by measuring transmittance in a particular wavelength or through colorimetric analysis.

In detail, in measuring the density of creatinine in urine, first, the picric acid is mixed in an alkaline solution (e.g., NaOH aqueous solution), light transmittance measured and set as a reference value, a urine sample is additionally mixed in the mixture solution of the picric acid and the alkaline solution, and a change in the light transmittance is then measured, to thus measure the density of creatinine.

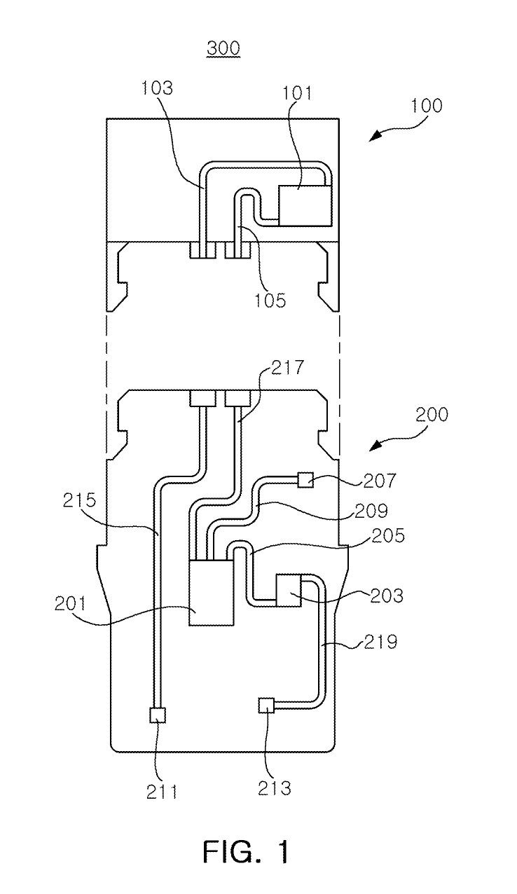

FIG. 1 is a view illustrating the structure of a cartridge for measuring a biological sample component according to an exemplary embodiment of the present invention.

With reference to FIG. 1, a cartridge 300 according to an exemplary embodiment of the present invention includes an upper cartridge 100 and a lower cartridge 200 bound to the upper cartridge 100 by an external force.

The upper cartridge 100 includes a first chamber 101 temporarily storing a retrieved (or collected) biological sample, a first channel 103 delivering air pressure provided from the lower cartridge 200 to the first chamber 101, and a third channel 105 transferring the biological sample temporarily stored in the first chamber 101 to the lower cartridge 200.

The lower cartridge 200 includes third and fifth chambers 201 and 203 for storing and mixing a diagnostic reagent, a fifth channel 205 installed between the third and fifth chambers 201 and 203 to transfer a diagnostic reagent, a first air exhaust hole 207 exhausting extra air present in the interior of the third chamber 201, and a seventh channel 209 connecting the first air exhaust hole 207 and the third chamber 201.

The lower cartridge 200 further includes first and third pump holes 211 and 213 for receiving air pressure from an external pump, a ninth channel 215 connected to the first pump hole 211 to transmit the air pressure provided through the first pump hole 211 to the first channel 103 of the upper cartridge 100, an eleventh channel 217 receiving a biological sample provided from the third channel 105 of the upper cartridge 100 and delivering the received biological sample to the third chamber 201, and a thirteenth channel 219 connecting the third pump hole 213 and the fifth chamber 203 and delivering air pressure provided from an external pump through the third pump 213 to the fifth chamber 203.

In the cartridge 300 according to an exemplary embodiment of the present invention illustrated in FIG. 1, a connection member (e.g., an O-ring) made of rubber or a silicon material is installed at a connection portion of each of the channels (e.g., the ninth channel 215 and the eleventh channel 217 or the first channel 103 and the third channel 105) connected between the upper cartridge 100 and the lower cartridge 200, in order to prevent a leakage of air pressure or a sample.

The cartridge 300 according to an exemplary embodiment of the present invention illustrated in FIG. 1 may be used to detect the density of one particular component among those included in a biological sample. For example, the cartridge 300 illustrated in FIG. 1 may be used to measure the density of creatinine included in urine.

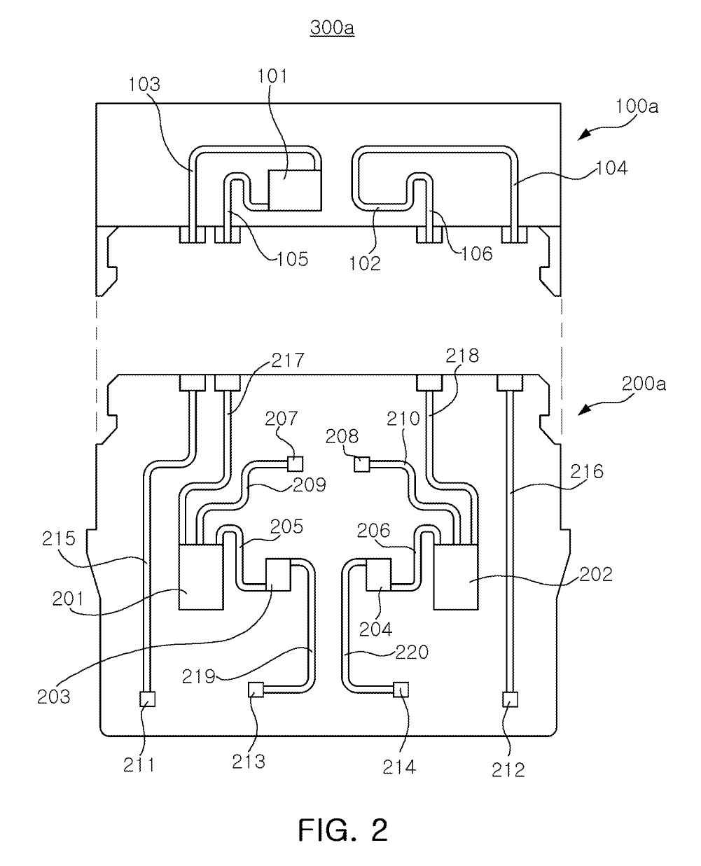

FIG. 2 is a view illustrating the structure of a cartridge for measuring a biological sample component according to another exemplary embodiment of the present invention. Specifically, FIG. 2 illustrates a cartridge structure for detecting the density of two particular components among those included in a biological sample.

With reference to FIG. 2, a cartridge 300a according to another exemplary embodiment of the present invention includes an upper cartridge 100a and a lower cartridge 200a bound to the upper cartridge 100a by an external force.

The upper cartridge 100a includes first and second chambers 101 and 102 temporarily storing a retrieved (or collected) biological sample, first and second channels 103 and 104 delivering air pressure provided from the lower cartridge 200a to the first and second chambers 101 and 102, and third and fourth channels 105 and 106 transferring the biological sample temporarily stored in the first and second chambers 101 and 102 to the lower cartridge 200a, respectively. Here, a urine sample may be temporarily stored in the first and second chambers 101 and 102.

The lower cartridge 200a includes a third chamber 201, a fourth chamber 202, a fifth chamber 203, and a sixth chamber 204 for storing and mixing a diagnostic reagent, a fifth channel installed between the third and fifth chambers 201 and 203 to transfer the diagnostic reagent, and a sixth chamber 206 installed between the fourth and sixth chambers 202 and 204 to transfer the diagnostic reagent. Here, at an early stage of measuring components of the biological sample, picric acid may be stored in the third chamber 202, an aqueous sodium hydroxide solution (NaOH) solution may be stored in the fifth chamber 203, a PVP aqueous solution may be stored in the fourth chamber 202, and an antibody directed against microalbumin may be stored in the sixth chamber 204.

The lower cartridge 200a further includes first and second air exhaust holes 207 and 208 exhausting extra air present in the interior of the third and fourth chambers 201 and 202, respectively, a seventh channel 209 connecting the first air exhaust hole 207 and the third chamber 201, and an eighth channel 210 connecting the second air exhaust hole 208 and the fourth chamber 202.

The lower cartridge 200a further includes first to fourth pump holes 211 to 214 delivering air pressure from an external pump hole, and ninth and tenth channels 215 and 216 connected to the first and second pump holes 211 and 212 to transmit air pressure provided through the first and second pump holes 211 and 212 to the first and second channels 103 and 104 of the upper cartridge 100a, respectively, eleventh and twelfth channels 217 and 218 receiving a biological sample provided from the third and fourth channels 106 of the upper cartridge 100a and delivering the received biological channel to the third and fourth chambers 201 and 202, respectively, a thirteenth channel 209 connecting the third pump 213 and the fifth chamber 203 to deliver air pressure provided from an external pump through the third pump hole 213 to the fifth chamber 203, and a fourteenth channel 220 connecting the fourth pump hole 214 and the sixth chamber 204 to deliver air pressure provided from an external pump through the fourth pump hole 214 to the sixth chamber 204.

In the cartridge 300a according to another exemplary embodiment of the present invention illustrated in FIG. 2, a connection member (e.g., an O-ring) made of rubber or a silicon material is installed at a connection portion of each of the channels (e.g., the ninth channel 215, the tenth channel 216, the eleventh channel 217 and the twelfth channel 213, or the first channel 103, the second channel 102, the third channel 105, and the fourth channel 106) connected between the upper cartridge 100 and the lower cartridge 200, in order to prevent a leakage of air pressure or a sample.

The cartridge 300a according to another exemplary embodiment of the present invention illustrated in FIG. 2 may be used to detect the density of two types of particular components among those included in a biological sample. For example, the cartridge 300a illustrated in FIG. 2 may be used to simultaneously measure the density of creatinine and microalbumin included in urine.

The cartridges 300 and 300a illustrated in FIGS. 1 and 2 may be disposable.

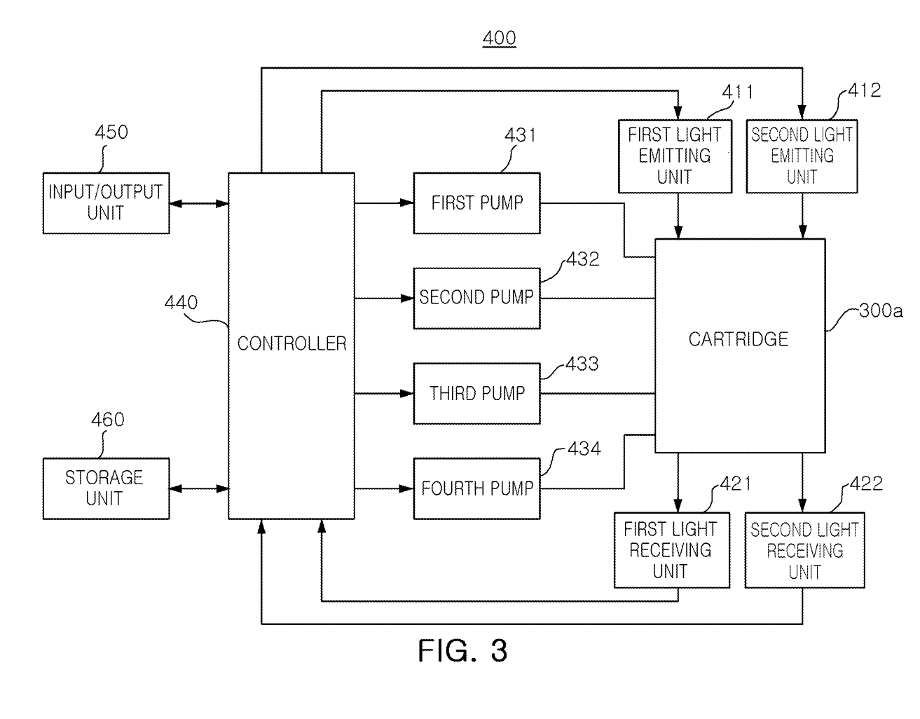

FIG. 3 is a schematic block diagram of the apparatus for measuring a biological sample component according to an exemplary embodiment of the present invention. Specifically, FIG. 3 illustrates an apparatus for detecting the density of a particular component included in a biological sample by using the cartridge 300a illustrated in FIG. 2. Here, it is assumed that the cartridge 300a is configured by binding the upper cartridge 100a and the lower cartridge 200a, a biological sample is stored in the first and second chambers 101 and 102, and a diagnostic reagent is stored in the third chamber 201, the fourth chamber 202, the fifth chamber 203, and the sixth chamber 204 of the lower cartridge 200a.

With reference to FIG. 3, the biological sample component measurement apparatus 400 according to an exemplary embodiment of the present invention includes a first light emitting unit 411, a second light emitting unit 412, a first light receiving unit 421, a second light receiving unit 422, a first pump 431, a second pump 432, a third pump 433, a fourth pump 434, and a controller 440. The biological sample component measurement apparatus 400 further includes an input/output unit 450 and a storage unit 460 as necessary.

The first light emitting unit 411, which may be configured as a light emitting diode (LED), is activated under the control of the controller 440 to emit light to the third chamber 201.

Here, at an initial stage, picric acid is stored in the third chamber 201, and afterwards, a mixture of the picric acid and a NaOH aqueous solution is stored in the third chamber 201 in order to a reference value of the amount of transmitted light in the process of measuring the density of creatinine, and thereafter, a mixture obtained by mixing a urine sample to the foregoing mixture is stored in the third chamber 201 in order to measure the density of creatinine included in urine.

The second light emitting unit 412, which may be configured as an LED, is activated under the control of the controller 440 to emit light to the fourth chamber 202 of the cartridge 300a.

Here, at the initial stage, a PVP aqueous solution is stored in the fourth chamber 202, and afterwards, a mixture of the PVP aqueous solution and a urine sample is stored in order to set a reference value of the amount of transmitted light in the process of measuring the density of microalbumin, and thereafter, a mixture obtained by mixing an antibody directed against microalbumin to the mixture of the PVP aqueous solution and the urine sample is stored in order to measure the density of microalbumin in the urine sample.

The first light receiving unit 421, which may be configured as a light receiving element such as a photodiode, or the like, detects light which has transmitted through the third chamber 201 of the cartridge 300a, and provides an electrical signal corresponding to the detected light to the controller 440.

The second light receiving unit 422, which may be configured as a light receiving element such as a photodiode, or the like, detects light which has transmitted through the fourth chamber 202 of the cartridge 300a, and provides an electrical signal corresponding to the detected light to the controller 440.

The first pump 431, which is connected to the first pump hole 211 of the cartridge 300a, activated by a control signal from the controller 440 to provide air pressure having a pre-set pressure level to the first pump hole 211. As discussed above, the air pressure provided by the first pump 431 is delivered to the first chamber 101 through the first channel 103 of the upper cartridge 100a connected to the ninth channel 215 of the lower cartridge 200a to force a biological sample temporarily stored in the first chamber 101 to be transferred to the third chamber 201 through the third channel 105 and the eleventh channel 217.

The second pump 432, which is connected to the second pump hole 212 of the cartridge 300a, is activated by a control signal from the controller 440 to provide air pressure of a pre-set size to the second pump hole 212. As discussed above, the air pressure provided by the second pump 432 is delivered to the second chamber 102 through the second channel 104 of the upper cartridge 100a connected to the tenth channel 216 of the lower cartridge 200a to force a biological sample temporarily stored in the second chamber 102 to be transferred to the fourth chamber 202 through the fourth channel 106 and the twelfth channel 218.

The third pump 433, which is connected to the third pump hole 213 of the cartridge 300a, is activated by a control signal from the controller 440 to provide air pressure having a pre-set pressure level to the third pump hole 213. As discussed above, the air pressure provided by the third pump 433 is delivered to the fifth chamber 203 through the thirteenth channel 219 of the lower cartridge 200a to force a diagnostic reagent (e.g., a NaOH aqueous solution) stored in the fifth chamber 203 to be transferred to the third chamber 201 through the fifth channel 205 and mixed with a material (e.g., picric acid) stored in the third chamber 201.

The fourth pump 434, which is connected to the fourth pump hole 214 of the cartridge 300a, is activated by a control signal from the controller 440 to provide air pressure of a pre-set size to the fourth pump hole 214. As discussed above, the air pressure provided by the fourth pump 434 is delivered to the sixth chamber 204 through the fourteenth channel 220 of the lower cartridge 200a to force a diagnostic reagent (e.g., an antibody directed against microalbumin) stored in the sixth chamber 204 to be transferred to the fourth chamber 202 through the sixth channel 206 and mixed with a material (e.g., a PVP aqueous solution) stored in the fourth chamber 202.

In a state in which the first to fourth pumps 431 to 434 are connected to the first to fourth pump holes 211 to 214 of the cartridge 300a, when an event signal instructing the measurement of a component of a biological sample is provided, the controller 440 controls the driving of the first to fourth pumps 431 to 434 to transfer and mix a diagnostic reagent or the biological sample, and controls the driving of the first and second light emitting units 411 and 412 to measure the density of two particular components (e.g., microalbumin and creatinine) included in the biological sample (e.g., a urine sample) on the basis of light signals provided from the first and second light receiving units.

In detail, in order to measure the density of creatinine in the urine sample, the controller 440 controls the driving of the third pump 433 to provide air pressure of a pre-set size to the third pump hole 213 to force the NaOH aqueous solution stored in the fifth chamber 203 to be transferred to the third chamber 201 and mixed with the picric acid stored in the third chamber 201.

Subsequently, when a pre-set period of time (namely, an amount of time required for the mixing of the NaOH aqueous solution and the picric acid) lapses, the controller 440 drives the first light emitting unit 411 and sets a first reference value of the amount of transmitted in order to measure the density of creatinine included in the urine sample on the basis of a light reception signal provided from the first light receiving unit 421.

Thereafter, the controller 440 controls the driving of the first pump 431 to force the urine sample stored in the first chamber 101 of the cartridge 300a to be transferred to the third chamber 201 of the cartridge 300a, and then, when a pre-set mixture time lapses, the controller 440 drives the first light emitting unit 411, receives a light reception signal corresponding to the amount of transmitted light from the first light receiving unit 421, calculates a variation of the amount of transmitted light by comparing the first reference value and the amount of transmitted light provided from the first light receiving unit 421, and then measures the density of creatinine included in the biological sample on the basis of the calculated variation of the amount of transmitted light.

Here, the controller 440 may display the measured density of creatinine through the input/output unit 450 or store the same in the storage unit 460.

In addition, in order to measure the density of microalbumin in the urine sample, the controller 440 controls the driving of the second pump 432 to provide air pressure of a pre-set size to the second pump hole 212 to force the urine sample stored in the second chamber 120 to be transferred to the fourth chamber 202 so as to be mixed with the PVP aqueous solution stored in the fourth chamber 202.

Subsequently, when a pre-set period of time (namely, an amount of time required for the mixing of the urine sample and the PVP aqueous solution and the picric acid) lapses, the controller 440 drives the second light emitting unit 412 and sets a second reference value of the amount of transmitted light in order to measure the density of microalbumin included in the urine sample on the basis of a light reception signal provided from the second light receiving unit 422.

Thereafter, the controller 440 controls the driving of the fourth pump 434 to force the antibody directed against microalbumin stored in the sixth chamber 204 to be transferred to the fourth chamber 202, and then, when a pre-set mixture time lapses, the controller 440 drives the second light emitting unit 412, receives a light reception signal corresponding to the amount of transmitted light from the second light receiving unit 422, calculates a variation of the amount of transmitted light by comparing the second reference value and the amount of transmitted light provided from the second light receiving unit 422, and then measures the density of microalbumin included in the biological sample on the basis of the calculated variation of the amount of transmitted light.

Here, the controller 440 may display the measured density of microalbumin through the input/output unit 450 or store the same in the storage unit 460.

In addition, the controller 440 may simultaneously measure the density of the creatinine and that of microalbumin in the urine sample as described above.

The input/output unit 450 may include a keypad and a display element for a touch screen), provide a key event signal corresponding to an input signal generated according to a user manipulation to the controller 440, and display measurement results, or the like, provided from the controller 440.

The storage unit 460 may be configured as a non-volatile memory in which data measured under the control of the controller 440 may be stored.

The biological sample component measurement apparatus 400 according to an exemplary embodiment of the present invention illustrated in FIG. 3 simultaneously measures the density of microalbumin and that of creatinine in urine, but in a different exemplary embodiment of the present invention, when the biological sample component measurement apparatus 400 employs the cartridge 300 illustrated in FIG. 1, the density of microalbumin and that of creatinine may be sequentially measured, and in this case, the second light emitting unit 412, the second light receiving unit 422, the second pump 432, and the fourth pump 434 may be omitted.

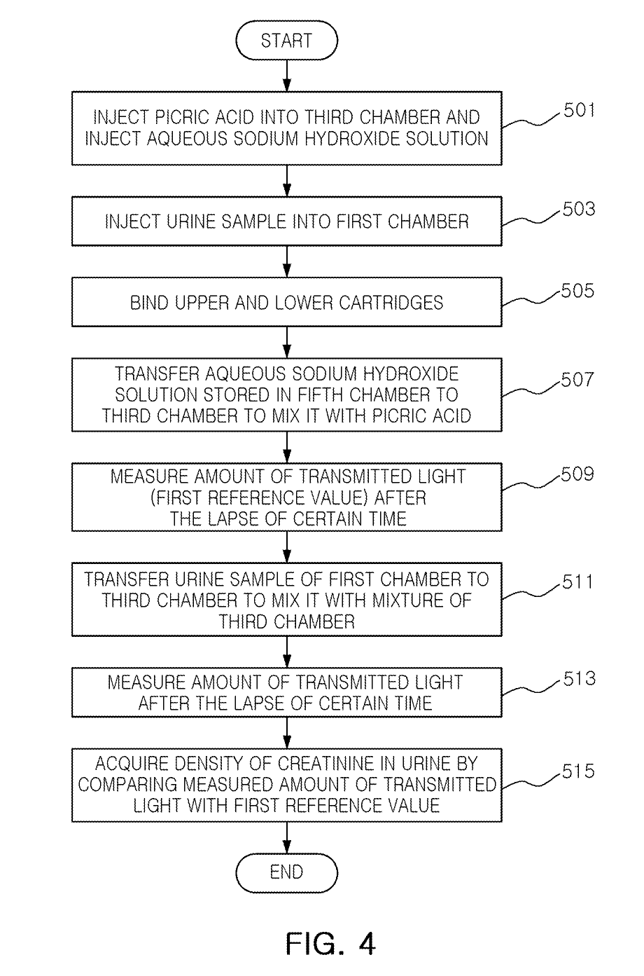

FIG. 4 is a flow chart illustrating the process of a method for measuring a biological sample component according to an exemplary embodiment of the present invention. Specifically, FIG. 4 shows the process of measuring the density of creatinine in a urine sample.

With reference to FIGS. 2 to 4, first, a user injects picric acid into the third chamber 201 of the lower cartridge 200a and then injects a NaOH aqueous solution into the fifth chamber 203 (step 501).

Also, the user injects a urine sample into the first chamber 101 of the upper cartridge 100a (step 503). Here, step 501 and step 503 may be performed in the reverse order.

After the urine sample and a diagnostic reagent are injected into the corresponding chambers, the upper cartridge 100a and the lower cartridge 200a are bound (step 505), and the first pump hole 211 to fourth pump hole 214 of the cartridge 300a are connected to the first pump 431 to fourth pump 434 of the biological sample component measurement apparatus 400, respectively.

Then, the controller 440 of the biological sample component measurement apparatus 400 drives the third pump 433 to provide air pressure to the fifth chamber 203 to force the NaOH aqueous solution stored in the fifth chamber 203 to be transferred to the third chamber 201 so as to be mixed with the picric acid stored in the third chamber 201 (step 507).

Thereafter, the controller 440 waits for a certain time required for the mixing of the NaOH aqueous solution and the picric acid, drives the first light emitting unit 411 to irradiate light to the third chamber 201, measures the amount of light transmitted through the third chamber 201, and sets the measured amount of transmitted light as a first reference value (step 509).

Subsequently, the controller 440 drives the first pump 431 to transfer the urine sample stored in the first chamber 101 to the third chamber 201 so that the urine sample can be mixed with a mixture stored in the third chamber 201 (step 511).

Thereafter, the controller 440 waits for a certain time required for the mixing operation, drives the first light emitting unit 411 to irradiate light to the third chamber 201, and measures the amount of light transmitted through the third chamber 201 (step 513).

Then, the controller 440 compares the first reference value which has been set in step 509 and the amount of transmitted light measured in step 513 to calculate the density of creatinine in urine (step 515).

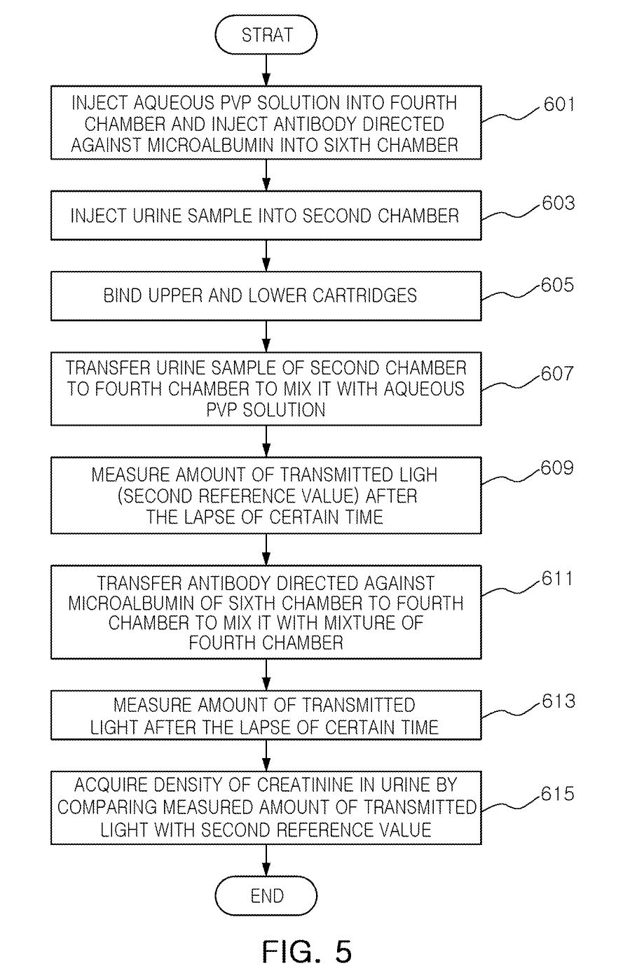

FIG. 5 is a flow chart illustrating the process of a method for measuring a biological sample component according to another exemplary embodiment of the present invention. Specifically, FIG. 5 shows the process of measuring the density of microalbumin in a urine sample.

With reference to FIGS. 2, 3, and 5, first, the user injects a PVP aqueous solution into the fourth chamber 202 of the lower cartridge 200a and also injects an antibody directed against microalbumin into the sixth chamber 204 (step 601).

Also, the user injects a urine sample into the second chamber 102 of the upper cartridge 100a (step 603). Here, steps 601 and 603 may be performed in the reverse order.

After the urine sample and a diagnostic reagent are injected into the corresponding chambers, the upper cartridge 100a and the lower cartridge 200a are bound (step 605), and the first pump hole 211 to fourth pump hole 214 of the cartridge 300a are connected to the first pump 431 to fourth pump 434 of the biological sample component measurement apparatus 400, respectively.

Then, the controller 440 of the biological sample component measurement apparatus 400 drives the second pump 432 to provide air pressure to the second chamber 102 to force the urine sample stored in the second chamber 102 to be transferred to the fourth chamber 202 so as to be mixed with the PVP aqueous solution stored in the fourth chamber 202 (step 607).

Thereafter, the controller 440 waits for a certain time required for the mixing of the urine sample and the PVP aqueous solution, drives the second light emitting unit 412 to irradiate light to the fourth chamber 202, measures the amount of light transmitted through the fourth chamber 202, and sets the measured amount of transmitted light as a second reference value (step 609).

Subsequently, the controller 440 drives the fourth pump 434 to transfer the antibody directed against microalbumin stored in the sixth chamber 204 to the fourth chamber 202 so that the antibody directed against microalbumin can be mixed with a mixture stored in the fourth chamber 202 (step 611).

Thereafter, the controller 440 waits for a certain time required for the mixing operation, drives the second light emitting unit 412 to irradiate light to the fourth chamber 202, and measures the amount of light transmitted through the fourth chamber 202 (step 613).

Then, the controller 440 compares the second reference value which has been set in step 609 and the amount of transmitted light measured in step 613 to calculate the density of microalbumin in urine (step 615).

As set forth above, in the cartridge for measuring a biological sample and the apparatus for measuring a biological sample according to exemplary embodiments of the invention, the urine sample and a diagnostic reagent for measuring the density of microalbumin and creatinine in the urine sample are stored by using the cartridge, and then, the apparatus for measuring a biological sample is connected to the cartridge in order to mix the urine sample and the diagnostic reagent stored in the cartridge and measures absorbance of the mixture to measure the density of the microalbumin and creatinine in the urine sample.

Thus, the density of the microalbumin and creatinine in the urine can be easily measured, whereby the user can easily check whether or not his kidney functions are abnormal and check the state of his kidney.

In addition, the cartridge and the apparatus for measuring a biological sample component can be widely utilized in fields such as the chemical, biological and medical fields, or the like, requiring the mixing of a biological sample and one or two or more diagnostic reagents.

While the present invention has been shown and described in connection with the exemplary embodiments, it will be apparent to those skilled in the art that modifications and variations can be made thereto without departing from the spirit and scope of the invention as defined by the appended claims.

What is claimed is:

1. A cartridge for measuring a biological sample component, the cartridge comprising:
an upper cartridge having a first chamber storing a biological sample, a first channel connected to the first chamber and delivering air pressure to the first chamber, and a third channel transferring the biological sample; and
a lower cartridge having a third chamber storing a first reagent, a fifth chamber storing a second reagent, a fifth channel connecting the third and fifth chambers and transferring the second reagent to the third chamber, an eleventh channel delivering the biological sample delivered through the third channel to the third chamber, and a thirteenth channel delivering air pressure to the fifth chamber,
wherein when the upper and lower cartridges are bound by an external force, the third channel and the eleventh channel are connected.

2. The cartridge of claim 1, wherein the lower cartridge further comprises a ninth channel connected to the first channel when the lower cartridge is bound to the upper cartridge, in order to provide air pressure provided from the exterior to the first channel.

3. The cartridge of claim 2, wherein first and third pump holes are prepared in one side of the ninth channel and in one side of the thirteenth channel and are connected to an external pump, respectively, in order to be provided with air pressure therethrough, respectively.

4. The cartridge of claim 1, wherein the lower cartridge further comprises a first air exhaust hole connected to the third chamber to exhaust air present in the third chamber and a seventh channel connecting the first air exhaust hole and the third chamber.

5. The cartridge of claim 1, wherein the upper cartridge further comprises a second chamber storing the biological sample, a second channel connected to the second chamber to deliver air pressure to the second chamber, and a fourth channel transferring the biological sample, and
the lower cartridge further comprises a fourth chamber storing a third reagent, a sixth chamber storing a fourth reagent, a sixth channel connecting the fourth and sixth chambers and transferring the fourth reagent to the fourth chamber, a twelfth channel delivering the biological sample delivered through the fourth channel to the fourth chamber, and a fourteenth channel delivering air pressure to the sixth chamber.

6. The cartridge of claim 5, wherein the lower cartridge further comprises a tenth channel connected to the second channel when the lower cartridge is bound to the upper cartridge to provide air pressure provided from the exterior to the second channel.

7. The cartridge of claim 5, wherein second and fourth pump holes are prepared in one side of the tenth channel and in one side of the fourteenth channel and are connected to an external pump, respectively, in order to be provided with air pressure, respectively.

8. The cartridge of claim 1, wherein the lower cartridge further comprises a second air exhaust hole connected to the fourth chamber and exhausting air present in the fourth chamber, and a sixth channel connecting the second air exhaust hole and the fourth chamber.

9. An apparatus for measuring a biological sample component using a cartridge storing a biological sample and at least one reagent and a plurality of channels transferring the biological sample and the at least one reagent and delivering air pressure, the apparatus comprising:
a first pump providing a first air pressure for transferring a biological sample stored in a first chamber of the cartridge to a third chamber of the cartridge, to a channel connected to the first chamber;
a third pump providing a second air pressure for transferring a second reagent stored in a fifth chamber of the cartridge to a third chamber of the cartridge, to a channel connected to the fifth chamber;
a first light emitting unit emitting light to a mixture stored in the third chamber;
a first light receiving unit detecting the amount of light which has transmitted through the mixture stored in the third chamber and providing the detected amount of light; and
a controller controlling the driving of the first and third pumps to allow the biological sample and the at least one reagent to be mixed in the third chamber, and acquiring the density of a first component of the biological sample.

10. The apparatus of claim 9, wherein the controller controls the driving of the third pump to transfer the second reagent stored in the fifth chamber to the third chamber to mix the second reagent and the first reagent stored in the third chamber, and then controls the driving of the first light emitting unit to emit light to the third chamber and set a first amount of transmitted light provided from the first light receiving unit as a first reference value for measuring the density of the first component of the biological sample.

11. The apparatus of claim 10, wherein, after setting the first reference value, the controller controls the driving of the first pump to transfer the biological sample stored in the first chamber to the third chamber to mix the biological sample and the mixture which has been obtained by mixing the first and second reagents, and then controls the driving of the first light emitting unit to emit light to the third chamber, acquire a second amount of transmitted light provided from the first light emitting unit, and measure the density of the first component by comparing the acquired second amount of transmitted light and the first reference value.

12. The apparatus of claim 11, wherein the biological sample is a urine sample, the first reagent is an picric acid, the second reagent is an aqueous sodium hydroxide solution (NaOH solution), and the first component is creatinine.

13. The apparatus of claim 9, further comprising:
a second pump providing a third air pressure, which is for transferring the biological sample stored in the second chamber of the cartridge to the fourth chamber of the cartridge, to a channel connected to the second chamber;
a fourth pump providing a fourth air pressure, which is for transferring the third reagent stored in the sixth chamber to the fourth chamber of the cartridge, to a channel connected to the sixth chamber;
a second light emitting unit emitting light to a mixture stored in the fourth chamber; and
a second light receiving unit detecting the amount of light which has transmitted through the mixture stored in the fourth chamber and providing the detected amount of light,
wherein the controller controls the driving of the second and fourth pumps to allow the biological sample and the at least one reagent to be mixed in the fourth chamber, and acquires the density of a second component of the biological sample.

14. The apparatus of claim 13, wherein the controller controls the driving of the second pump to transfer the biological sample stored in the second chamber to the fourth chamber to mix the biological sample and the third reagent stored in the fourth chamber, and then controls the driving of the second light emitting unit to emit light to the fourth chamber and set a third amount of transmitted light provided from the second light receiving unit as a second reference value for measuring the density of the second component of the biological sample.

15. The apparatus of claim 14, wherein, after setting the second reference value, the controller controls the driving of the fourth pump to transfer the fourth reagent stored in the sixth chamber to the fourth chamber to mix the fourth reagent and the mixture which has been obtained by mixing the biological sample and the third reagent, and then controls the driving of the second light emitting unit to emit light to the fourth chamber, acquire a fourth amount of transmitted light provided from the second light emitting unit, and measure the density of the second component by comparing the acquired fourth amount of transmitted light and the second reference value.

16. The apparatus of claim 15, wherein the biological sample is a urine sample, the third reagent is an aqueous polyvinylpyrolidone (PVP) solution, the fourth reagent is an antibody directed against microalbumin, and the second component is microalbumin.
